(19) United States
(12) Patent Application Publication
(10) Pub. No.: US 2019/0076606 A1
(43) Pub. Date: Mar. 14, 2019

(54) NEBULIZER

(71) Applicant: HCMED INNOVATIONS CO., LTD, TAIPEI CITY (TW)

(72) Inventors: KE-TING CHEN, TAIPEI CITY (TW); WEN-YU TSAI, TAIPEI CITY (TW); CHIEH-SHENG CHENG, TAIPEI CITY (TW)

(21) Appl. No.: 15/893,734

(22) Filed: Feb. 12, 2018

(30) Foreign Application Priority Data
Sep. 13, 2017 (TW) 106131387

(51) Int. Cl. A61M 11/00

(52) U.S. Cl. CPC A61M 11/00; A61M 2205/3313; A61M 2205/3334; A61M 2205/702

(57) ABSTRACT

A nebulizer includes a nebulizer main body, an atomization module, a mouthpiece, a signal transmitter and a signal sensor. The atomization module is disposed in the nebulizer main body, and has a plurality of atomization holes. The mouthpiece is disposed on the nebulizer main body, and has an opening corresponding to the plurality of atomization holes. The signal transmitter provides a sensing signal toward the plurality of atomization holes of the atomization module. The signal sensor corresponds to the signal transmitter. The sensing signal passes through or is reflected by the atomization module, so as to produce a clogging rate signal. The signal sensor then receives the clogging rate signal to determine the clogging rate of the plurality of atomization holes.

### FIELD OF THE INVENTION

The present disclosure relates to a nebulizer, in particular to a nebulizer that can determine a clogging rate of an atomization module.

### BACKGROUND OF THE INVENTION

For the existing nebulizer, a micro-hole structure therein used for transforming a liquid into droplets and spraying the droplets, will be gradually clogged after long-term use. When this occurs, a user would need to disassemble the nebulizer to check the clogging state of the micro-hole structure of the nebulizer, so as to clean the micro-hole structure. A microscopic or optical device may be used to check the clogging state of the micro-hole structure. However, disassembly of the nebulizer may cause damage to elements of the nebulizer, greatly inconveniencing users thereof.

### SUMMARY OF THE INVENTION

The object of the present disclosure is to provide a nebulizer that can determine a clogging rate of an atomization module to solve a problem of the prior art.

Accordingly, an embodiment of the present disclosure provides a nebulizer. The nebulizer includes a nebulizer main body, an atomization module, a mouthpiece, a signal transmitter, and a signal sensor. The atomization module is disposed in the nebulizer main body, and has a plurality of atomization holes. The mouthpiece is disposed on the nebulizer main body, and has an opening corresponding to the plurality of the atomization holes. The signal transmitter provides a sensing signal toward the plurality of the atomization holes of the atomization module. The signal sensor corresponds to the signal transmitter. The sensing signal provided by the signal transmitter passes through or is reflected by the atomization module so as to produce a clogging rate signal, the signal sensor receives the clogging rate signal to determine a clogging rate of the plurality of the atomization holes.

Accordingly, another embodiment of the present disclosure provides a nebulizer. The nebulizer includes a nebulizer main body, an atomization module, a mouthpiece, a signal transmitter, and a signal sensor. The atomization module is disposed in the nebulizer main body, and has a plurality of atomization holes. The mouthpiece is disposed on the nebulizer main body, and has an opening corresponding to the plurality of the atomization holes. The signal transmitter provides a sensing signal toward the plurality of the atomization holes of the atomization module. The signal sensor corresponds to the signal transmitter. In the atomization module, a clogging rate of the plurality of the atomization holes is determined by the signal transmitter in cooperation with the signal sensor.

The nebulizer provided by the present disclosure may determine the clogging rate of the plurality of the atomization holes by having the sensing signal provided by the signal transmitter passes through or be reflected by the atomization module to produce a clogging rate signal, or by using the signal transmitter in cooperation with the signal sensor.

To further understand the techniques, means and effects of the present disclosure, the following detailed descriptions and appended drawings are hereby referred to, such that, and through which, the purposes, features and aspects of the present disclosure can be thoroughly and concretely appreciated. However, the appended drawings are provided solely for reference and illustration, without any intention to limit the present disclosure.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 is a schematic view of a nebulizer according to a first embodiment of the present disclosure.

FIG. 2 is a schematic view of a nebulizer according to a second embodiment of the present disclosure.

FIG. 3 is a schematic view of a nebulizer according to a third embodiment of the present disclosure.

FIG. 4 is a schematic view of a nebulizer according to a fourth embodiment of the present disclosure.

### DETAILED DESCRIPTION OF THE PREFERRED EMBODIMENTS

Embodiments of a nebulizer according to the present disclosure are described herein. Other advantages and objectives of the present disclosure can be easily understood by one skilled in the art from the disclosure. The present disclosure can be applied in different embodiments. Various modifications and variations can be made to various details in the description for different applications without departing from the scope of the present disclosure. The drawings of the present disclosure are provided only for simple illustrations, but are not drawn to scale and do not reflect the actual relative dimensions. The following embodiments are provided to describe in detail the concept of the present disclosure, and are not intended to limit the scope thereof in any way.

The object of the present disclosure is to provide a nebulizer that can determine a clogging rate of an atomization module, so as to improve its convenience.

The present disclosure provides a nebulizer D, which includes a nebulizer main body 1, an atomization module 2, a mouthpiece 3, a signal transmitter 4 and a signal sensor 5. The atomization module 2 is disposed in the nebulizer main body, and the atomization module 2 has a plurality of atomization holes 201. The mouthpiece 3 is disposed on the nebulizer main body 1, and the mouthpiece 3 has an opening 301 corresponding to the plurality of atomization holes. The signal transmitter 4 provides a sensing signal toward the atomization module 2. The signal sensor 5 may be disposed to correspond to the signal transmitter 4. In the atomization module 2, a clogging state of the plurality of atomization holes 201 may be determined by the cooperation of the signal transmitter 4 and the signal sensor 5. Alternatively, the signal transmitter 4 may provide the sensing signal, and the sensing signal may pass through the atomization module 2, or may be reflected by the atomization module 2, so as to produce a clogging rate signal. The signal sensor 5 receives the clogging rate signal to determine the clogging rate of the plurality of atomization holes 201.

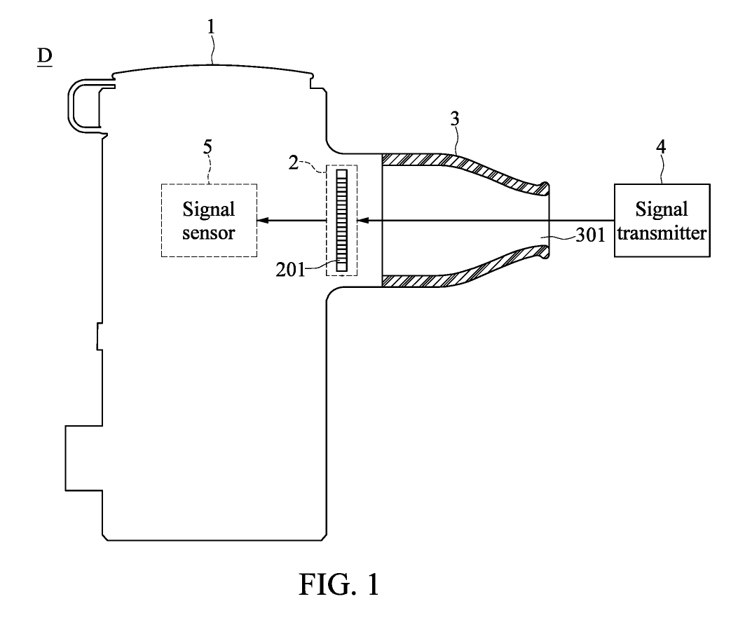

Reference is made to FIG. 1, which is a schematic view of a nebulizer D according to a first embodiment of the present disclosure. The present disclosure provides a nebulizer D, which includes the nebulizer main body 1, the atomization module 2, the mouthpiece 3, the signal transmitter 4 and the signal sensor 5.

In detail, the nebulizer main body 1 forms a main body of the nebulizer D. In practice, the nebulizer main body 1 may include an accommodating part and a body part, the accommodating part accommodating an active ingredient of a medicine, and the body part including an electronic element to electrically control enabling or disabling of the nebulizer D. The elements included in the nebulizer main body 1 of the present disclosure may be changed according to actual implementation for equivalent performance. It should be noted that, the shapes of the nebulizer D and the nebulizer main body 1 shown in FIG. 1 are used for descriptive purposes only, and the present disclosure is not limited thereto.

Furthermore, the atomization module 2 is disposed in the nebulizer main body 1. The atomization module 2 has a plurality of atomization holes 201. In actual implementation, the electronic element of the body part may be used to electrically control the enabling and disabling of the atomization module 2. When the atomization module 2 is used, the atomization module 2 atomizes the medicine in the accommodating part.

As mentioned above, the mouthpiece 3 is disposed on the nebulizer main body 1. As shown in FIG. 1, the mouthpiece 3 is disposed on the nebulizer main body 1, and disposed corresponding to a position of the atomization module 2. The mouthpiece 3 has an opening 301, when the mouthpiece 3 is disposed on the nebulizer main body 1, the opening 301 is disposed corresponding to positions of the plurality of atomization holes 201 of the atomization module 2.

The nebulizer D provided by the present disclosure further includes the signal transmitter 4 and the signal sensor 5. Generally, the signal transmitter 4 and the signal sensor 5 can cooperate with each other for determining a clogging state of the plurality of atomization holes 201 of the atomization module 2. Specifically, the signal transmitter 4 provides a sensing signal toward the atomization module 2. A clogging rate signal is obtained after the sensing signal passes through the atomization module 2, and the signal sensor 5 receives the clogging rate signal to determine the clogging rate of the plurality of atomization holes 201.

It should be noted that, the disposition of the signal transmitter 4 and the signal sensor 5 may be varied in different configurations of the present disclosure. The signal transmitter 4 and the signal sensor 5 may be disposed on a same side of the atomization module 2, or the signal transmitter 4 and the signal sensor 5 may be respectively disposed on two sides of the atomization module 2. It should be noted that, when the signal transmitter 4 and the signal sensor 5 are disposed on the same side of the atomization module 2, the signal transmitter 4 and the signal sensor 5 may form a complete module, such as a sensing module, but the present disclosure is not limited thereto.

As shown in FIG. 1, in the first embodiment of the present disclosure, the signal transmitter 4 and the signal sensor 5 of the nebulizer D are respectively disposed on two sides of the atomization module 2. More specifically, the signal transmitter 4 is disposed outside of the nebulizer D, and is preferably disposed outside of the mouthpiece 3. The signal sensor 5 is disposed in the nebulizer main body 1, so that the atomization module 2 is disposed between the signal transmitter 4 and the signal sensor 5. The signal transmitter 4 transmits a sensing signal from outside of the mouthpiece 3, the sensing signal passes the opening 301 of the mouthpiece 3 directly toward the plurality of atomization holes 201 of the atomization module 2. The sensing signal passes through the plurality of atomization holes 201 and produces a clogging rate signal, and the signal sensor 5 then receives the clogging rate signal to determine the clogging rate of the plurality of atomization holes 201.

As mentioned above, the sensing signal may be an energy wave, a fluid, or a combination of the two. For example, the energy wave can include a visible light of a specific wavelength, a laser beam, an infrared and a sound wave, etc. The fluid may be a gas or a liquid with a predetermined energy and a specific momentum, such as a waterspout or an air flow. In the embodiment, the signal transmitter 4 transmits an initial fluid with a predetermined momentum. The initial fluid with the predetermined momentum may be transmitted by jetting with high speed and high pressure. When the initial fluid used as the sensing signal is transmitted toward the plurality of atomization holes 201 of the atomization module 2, a penetration rate of the plurality of atomization holes 201 may be used to determine an amount of the fluid passing through the atomization holes 201. If the nebulizer D has been initialized for use, the plurality of atomization holes 201 should be in an unclogged state, and a mass of the initial fluid should be substantially equal to that of the fluid passing through the plurality of atomization holes 201. Otherwise, after long-term usage and the plurality of atomization holes 201 of the nebulizer D is gradually clogged, a difference between the mass of the initial fluid and that of the fluid passing through the plurality of atomization holes 201 would be greater. A variation of the parameters between the fluid passing through the atomization holes 201 and the initial fluid may produce a clogging rate signal, which can then be transmitted toward the signal sensor 5. More specifically, the clogging rate signal may be a momentum variation between the initial fluid and the fluid passing through the plurality of atomization holes 201, and the flow variation or an impact (i.e., pressure) received on a sensing unit area of the signal sensor 5. For example, if the pressure received by the signal sensor 5 is smaller, it would signify that the clogging rate of the plurality of atomization holes 201 is greater. Therefore, the signal sensor 5 can determine the clogging rate of the plurality of atomization holes 201 by the clogging rate signal.

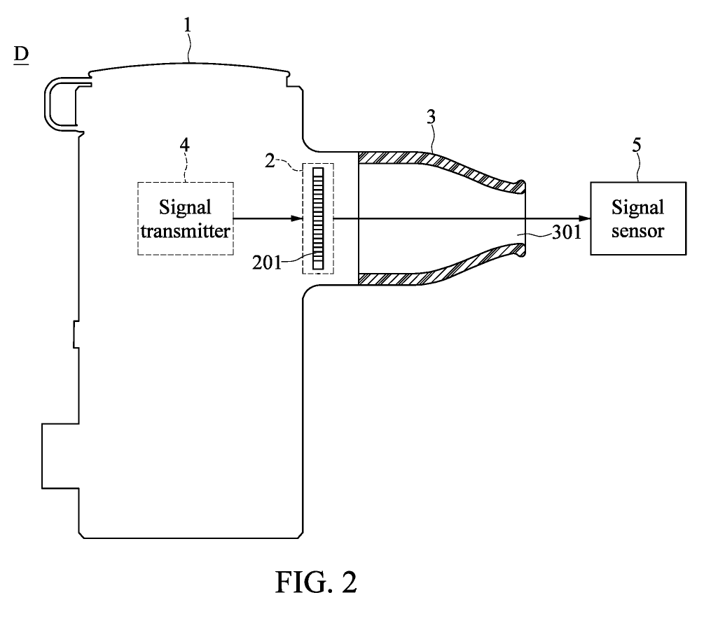

Reference is made to FIG. 2, which is a schematic view of a nebulizer D according to a second embodiment of the present disclosure. The present disclosure provides a nebulizer D, which includes the nebulizer main body 1, the atomization module 2, the mouthpiece 3, the signal transmitter 4 and the signal sensor 5.

The second embodiment differs from the first embodiment of the present disclosure in that, in the second embodiment, the signal sensor 5 is disposed outside of the nebulizer D, and is preferably disposed outside of the mouthpiece 3. The signal transmitter 4 is disposed in the nebulizer main body 1, so that the atomization module 2 is disposed between the signal transmitter 4 and the signal sensor 5.

The signal transmitter 4 provides the sensing signal from inside of the nebulizer main body 1 toward the atomization module 2. The sensing signal passes through the atomization module 2, and then produces a clogging rate signal. The signal sensor 5 receives the clogging rate signal to determine the clogging rate of the plurality of atomization holes 201.

Similar to the first embodiment, in the embodiment, the sensing signal may be an energy wave, a fluid, or a combination of the two. For example, the energy wave can include a visible light with a specific wavelength, a laser beam, a far infrared and a soundwave, and so on. The fluid may be given a predetermined energy and having a gas or a liquid with a specific momentum, such as a waterspout or an air flow. The working principle of this embodiment can be derived from the first embodiment, and further description thereon will be omitted herein.

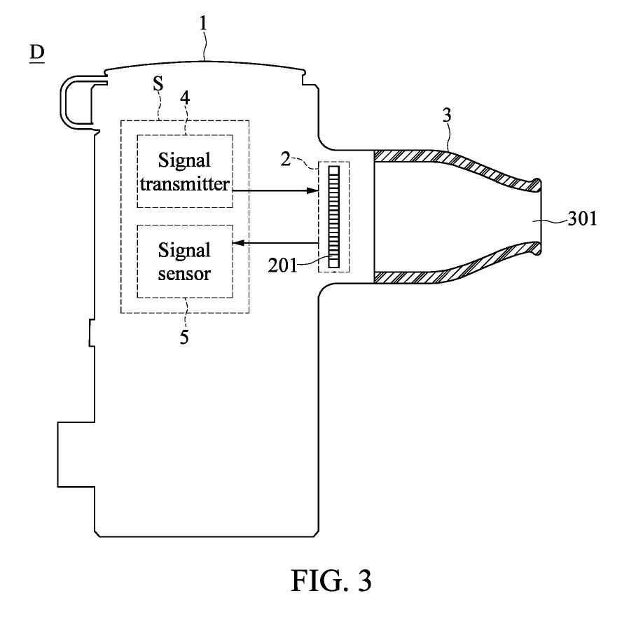

Reference is made to FIG. 3, which is a schematic view of a nebulizer D according to a third embodiment of the present disclosure. The present disclosure provides a nebulizer D, which includes the nebulizer main body 1, the atomization module 2, the mouthpiece 3, the signal transmitter 4 and the signal sensor 5.

In this embodiment, the signal transmitter 4 and the signal sensor 5 are both disposed in the nebulizer main body 1, and on a same side of the atomization module 2. In the embodiment, the signal transmitter 4 and the signal sensor 5 form a sensing module S. Similarly, the signal transmitter 4 transmits a sensing signal toward the atomization module 2, and the sensing signal is reflected by the atomization module 2 to produce a clogging rate signal. The signal sensor 5 receives the clogging rate signal to determine the clogging rate of the plurality of atomization holes 201 of the atomization module 2.

In detail, in the embodiment, the signal transmitter 4 transmits a laser light beam having a specific wavelength, such as a green laser light with a wavelength of 520 nm. The sensing signal (initial laser light) is projected toward the atomization module 2 and the plurality of atomization holes 201 thereof. The principle of this embodiment is using a laser reflection rate of the plurality of atomization holes 201 to produce the clogging rate signal. Specifically, if the nebulizer D has been initialized for use, then the plurality of atomization holes 201 would be unclogged, the laser reflection rate would be lower, an intensity of the reflection light would be smaller, and the clogging rate signal would be smaller. If the clogging rate signal received by the signal sensor 5 is smaller, then it may be determined that the clogging rate of the plurality of atomization holes 201 is smaller. Otherwise, after the nebulizer D is used for a period of time and the plurality of atomization holes 201 are gradually clogged, when the signal transmitter 4 transmits the laser beam toward the atomization module 2, the laser beam is reflected by the surface of the plurality of atomization holes 201, the intensity of the reflected light beam is used as the clogging rate signal, and the clogging rate signal can be received by the signal sensor 5 so as to determine the clogging state of the plurality of atomization holes 201.

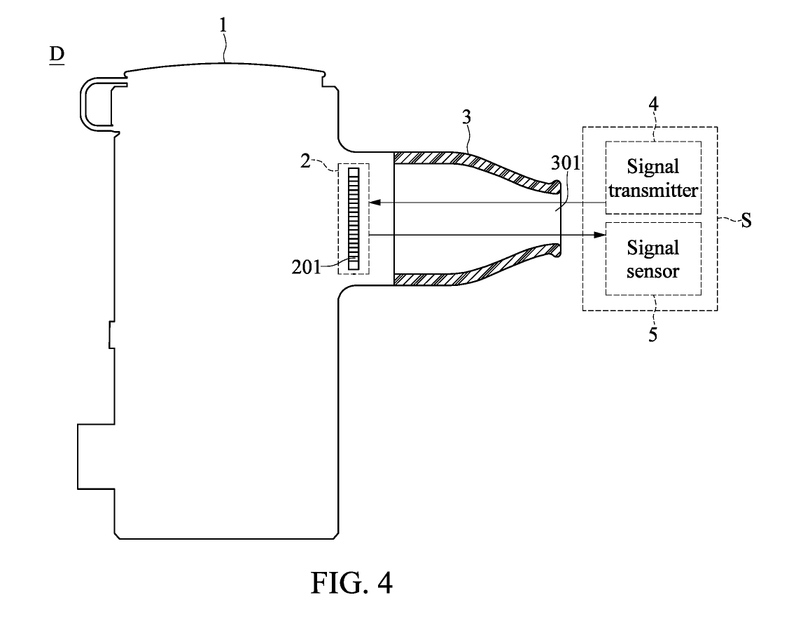

Reference is made to FIG. 4, which is a schematic view of a nebulizer D according to a fourth embodiment of the present disclosure. The present disclosure provides a nebulizer D, which includes the nebulizer main body 1, the atomization module 2, the mouthpiece 3, the signal transmitter 4 and the signal sensor 5.

In the embodiment, the signal transmitter 4 and the signal sensor 5 are both disposed on a same side of the atomization module 2. The fourth embodiment differs from the third embodiment of the present disclosure in that, in the fourth embodiment, the signal transmitter 4 and the signal sensor 5 are both disposed outside of the nebulizer D. In other words, the signal transmitter 4 and the signal sensor 5 are both outside of the mouthpiece 3, and are disposed to correspond to the position of the atomization module 2. Similarly, in the embodiment, the signal transmitter 4 and the signal sensor 5 may form a sensing module S. The signal transmitter 4 transmits the sensing signal to pass through an opening 301 of the mouthpiece 3 toward the atomization module 2. Specifically, the signal transmitter 4 transmits a green laser beam toward the plurality of atomization holes 201 of the atomization module 2. Similarly, the laser beam is reflected by the surface of the plurality of atomization holes 201 to form a clogging rate signal. The signal sensor 5 receives the clogging rate signal so as to determine the clogging rate of the plurality of atomization holes 201.

The present disclosure has the advantage that in the nebulizer D provided by the present disclosure, the clogging rate of the plurality of atomization holes 201 may be determined by producing a clogging rate signal using the sensing signal provided by the signal transmitter 4 that passes through or is reflected by the atomization module 2, to produce a clogging rate signal, or by using the signal transmitter 4 in cooperation with the signal sensor 5.

The aforementioned descriptions merely represent the preferred embodiments of the present disclosure, without any intention to limit the scope of the present disclosure which is fully described only within the following claims. Various equivalent changes, alterations or modifications based on the claims of the present disclosure are all, consequently, viewed as being embraced by the scope of the present disclosure.

What is claimed is:

1. A nebulizer comprising:
a nebulizer main body;
an atomization module disposed in the nebulizer main body, and having a plurality of atomization holes;
a mouthpiece disposed on the nebulizer main body, and having an opening corresponding to the plurality of the atomization holes;
a signal transmitter providing a sensing signal toward the plurality of the atomization holes of the atomization module; and
a signal sensor corresponding to the signal transmitter;
wherein the sensing signal provided by the signal transmitter passes through or is reflected by the atomization module so as to produce a clogging rate signal, the signal sensor receives the clogging rate signal to determine a clogging rate of the plurality of the atomization holes.

2. The nebulizer of claim 1, wherein one of the signal transmitter and the signal sensor is disposed in the nebulizer main body, and the other one of the signal transmitter and the signal sensor is disposed outside of the nebulizer main body, and the clogging rate signal is produced by having the sensing signal pass through the atomization module.

3. The nebulizer of claim 1, wherein the signal transmitter and the signal sensor are both disposed in the nebulizer main body and on a same side of the atomization module, and the sensing signal is reflected by the atomization module to produce the clogging rate signal.

4. The nebulizer of claim 1, wherein the signal transmitter and the signal sensor are both disposed outside of the nebulizer main body and on a same side of the atomization module, and the sensing signal is reflected by the atomization module to produce the clogging rate signal.

5. The nebulizer of claim 1, wherein the sensing signal is an energy wave, a fluid, or a combination thereof.

6. A nebulizer comprises:
a nebulizer main body;
an atomization module disposed in the nebulizer main body, and having a plurality of atomization holes;
a mouthpiece disposed on the nebulizer main body, and having an opening corresponding to the plurality of the atomization holes;
a signal transmitter providing a sensing signal toward the plurality of the atomization holes of the atomization module; and
a signal sensor corresponding to the signal transmitter;
wherein the atomization module is determined a clogging rate of the plurality of the atomization holes by the signal transmitter in cooperation with the signal sensor.

7. The nebulizer of claim 6, wherein one of the signal transmitter and the signal sensor is disposed in the nebulizer main body, and the other one of the signal transmitter and the signal sensor is disposed outside of the nebulizer main body, and the clogging rate signal is produced by having the sensing signal pass through the atomization module.

8. The nebulizer of claim 6, wherein the signal transmitter and the signal sensor are both disposed in the nebulizer main body and on a same side of the atomization module, and the sensing signal is reflected by the atomization module to produce the clogging rate signal.

9. The nebulizer of claim 6, wherein the signal transmitter and the signal sensor are both disposed outside of the nebulizer main body and on a same side of the atomization module, and the sensing signal is reflected by the atomization module to produce the clogging rate signal.

10. The nebulizer of claim 6, wherein the sensing signal is an energy wave, a fluid, or a combination thereof.
